(19) United States
(12) Patent Application Publication
(10) Pub. No.: US 2021/0015992 A1
(43) Pub. Date: Jan. 21, 2021

(54) SYSTEM AND METHOD FOR CARDIAC DIALYSIS

(71) Applicant: Break-Three Medical Industries, Inc, Dover, DE (US)

(72) Inventor: Doron WEINFELD, Jerusalem (IL)

(73) Assignee: Break-Three Medical Industries, Inc., Dover, DE (US)

(21) Appl. No.: 17/044,122

(22) PCT Filed: Apr. 1, 2019
(86) PCT No.: PCT/US2019/025200

§ 371 Date: Sep. 30, 2020

Related U.S. Application Data
(60) Provisional application No. 62/650,321, filed on Mar. 30, 2018.

(51) Int. Cl. A61M 1/36

(52) U.S. Cl. CPC A61M 1/3661; A61M 2202/0478; A61M 2205/0216; A61M 2210/125

(57) ABSTRACT

A system and method for treating a patient may include implanting an apparatus that includes a tube housing a one-way valve and having first and second surfaces. When the apparatus is implanted, the first surface is located inside the right atrium of the patient and the second surface is located on the skin of the patient. When implanted, the apparatus is used for drawing blood from the right atrium and/or providing a liquid to the patient. The apparatus may be designed to be hermetically sealed when disconnected from input and output lines.

### FIELD OF THE INVENTION

The present invention relates generally to treating a patient. More specifically, the present invention relates to directing blood flow from the right atrium of a patient to an external system and/or from an external system into the right atrium.

### BACKGROUND OF THE INVENTION

The need for extracting blood from a patient, treating the extracted blood and returning treated blood to the patient's blood system is known in the art, e.g., with respect to dialysis. End stage renal disease (ESRD) is considered the last stage of chronic kidney disease. Patients suffering from ESRD require either kidney transplant or dialysis in order to survive.

Generally, when performing dialysis, it is necessary to place a catheter within the central venous system. However, over time it may become impossible to place a catheter in the central veins of patients due to chronic thrombus formation and/or stenosis or narrowing or collapse of the central veins. For example, repeated venous access / dialysis venous access, may lead to chronic thrombus formation and/or stenosis, leading to failure of known endovascular methods to widen the veins (to enable insertion of a catheter).

Currently, when catheters can no longer be used, the patient must undergo kidney transplant. However, known systems and methods are helpless in situations where catheters can no longer be used and a kidney transplant is not an option.

Therefore, there is a need in the art for a method by which dialysis is performed without requiring traditional catheterization of the central venous system.

### SUMMARY OF THE INVENTION

An implantable apparatus for treating a patient may include a tube housing a one-way valve and having first and second surfaces. When the apparatus is implanted, the first surface is located inside the right atrium of the patient and the second surface is located on the skin of the patient and wherein, when implanted, the apparatus is used for drawing blood from the right atrium, treating the blood and providing a liquid to the patient. The apparatus may be designed to be hermetically sealed when disconnected from input and output lines.

The apparatus may be used for performing dialysis by: inserting a dialysis catheter through the one-way valve; performing dialysis by a system connected to the catheter; removing the dialysis catheter; and sealing the one-way valve. The first and second surfaces may be adapted to hold the tube in place by respectively applying pressure to the patient's skin and to the inner wall of the right atrium.

The apparatus may be implanted by: accessing the right atrium of the heart through an anterior chest wall of the patient, using a percutaneous needle; deploying a plurality of suture mounted anchors into the right atrium of the heart, through the percutaneous needle; anteriorly drawing the right atrium of the heart towards the anterior chest wall; inserting a portion of the apparatus into the right atrium of the heart along a guidewire; adjusting the distance between the first and second surfaces; tight locking the first and second surfaces in place; and removing the guidewire, the suture mounted anchors and the percutaneous needle.

The apparatus may include at least one elastic ring mounted on the tube and adapted to hold the conduit in place. The apparatus may be placed via one of: an upper extremity vein, a lower extremity vein, a direct inferior vena cava (IVC) puncture and an hepatic vein.

In some embodiments, a method of treating a patient may include: placing, in the right atrium of the patient, a device including at least one one-way valve; and using the device for drawing blood from the right atrium, treating the blood and providing a liquid to the patient. The method may include hermetically sealing the device when the device is disconnected from input and/or output lines. The method may include moving at least one elastic ring along a conduit included in the device to thereby hold the device in place. The method may include accessing the right atrium of the heart through an anterior chest wall of the patient, using a percutaneous needle; deploying a plurality of suture mounted anchors into the right atrium of the heart, through the percutaneous needle; anteriorly drawing the right atrium of the heart towards the anterior chest wall; inserting the device into the right atrium of the heart along a guidewire; adjusting the distance between the first and second surfaces; tightening locking the first and second surfaces in place; and removing the guidewire, the suture mounted anchors and the percutaneous needle.

In some embodiments, a method for performing dialysis via the one-way valve may include inserting a dialysis catheter into the one-way valve; performing dialysis by a system connected to the catheter; flushing the one-way valve device; removing the dialysis catheter; and sealing the one-way valve. The one-way valve may be made of several flexible overlapping diaphragms. The one one-way valve may be designed to be used with a dialysis catheter with an outer diameter between 2.5 mm to 5.5 mm.

In some embodiments, a device for treating a patient may be placed via one of: an upper extremity vein, a lower extremity vein, a direct IVC puncture and an hepatic vein. In some embodiments, a device for treating a patient may be placed in the right atrium using a micro-puncture access assembly. In some embodiments, a method of treating a patient may include implanting a device that includes a conduit, the conduit housing a one-way valve, such that one opening of the conduit is inside the right atrium of the patient and another opening of the conduit enables inserting a tube into the right atrium; and using the device for at least one of: drawing blood from the right atrium and providing a liquid to the patient. Other aspects and/or advantages of the present invention are described herein.

### BRIEF DESCRIPTION OF THE DRAWINGS

Non-limiting examples of embodiments of the disclosure are described below with reference to figures attached hereto that are listed following this paragraph. Identical features that appear in more than one figure are generally labeled with a same label in all the figures in which they appear. A label labeling an icon representing a given feature of an embodiment of the disclosure in a figure may be used to reference the given feature. Dimensions of features shown in the figures are chosen for convenience and clarity of presentation and are not necessarily shown to scale. For example, the dimensions of some of the elements may be exaggerated relative to other elements for clarity, or several physical components may be included in one functional block or element. Further, where considered appropriate, reference numerals may be repeated among the figures to indicate corresponding or analogous elements.

The subject matter regarded as the invention is particularly pointed out and distinctly claimed in the concluding portion of the specification. The invention, however, both as to organization and method of operation, together with objects, features and advantages thereof, may best be understood by reference to the following detailed description when read with the accompanied drawings. Embodiments of the invention are illustrated by way of example and not limitation in the figures of the accompanying drawings, in which like reference numerals indicate corresponding, analogous or similar elements, and in which:

FIG. 1 shows a device according to illustrative embodiments of the present invention;

FIG. 2 shows a one-way valve and a sealing apparatus according to illustrative embodiments of the present invention;

FIG. 3 shows an implanted device according to illustrative embodiments of the present invention;

FIG. 4 shows a device according to illustrative embodiments of the present invention; and

FIG. 5 shows a flowchart of a method according to illustrative embodiments of the present invention.

### DETAILED DESCRIPTION

In the following detailed description, numerous specific details are set forth in order to provide a thorough understanding of the invention. However, it will be understood by those skilled in the art that the present invention may be practiced without these specific details. In other instances, well-known methods, procedures, and components, modules and/or units have not been described in detail so as not to obscure the invention. Some features or elements described with respect to one embodiment may be combined with features or elements described with respect to other embodiments. For the sake of clarity, discussion of same or similar features or elements may not be repeated.

Although embodiments of the invention are not limited in this regard, the terms “plurality” and “a plurality” as used herein may include, for example, “multiple” or “two or more”. The terms “plurality” or “a plurality” may be used throughout the specification to describe two or more components, devices, elements, units, parameters, or the like. The term set when used herein may include one or more items.

Unless explicitly stated, the method embodiments described herein are not constrained to a particular order in time or chronological sequence. Additionally, some of the described method elements can occur or be performed simultaneously, at the same point in time, or concurrently, some of the described method elements may be skipped, or they may be repeated, during a sequence of operations of a method.

An embodiment may include implanting a tube that includes, or houses, one or more one-way valves such that blood from the right atrium of a patient can flow through a system that is external to the patient and back to the patient. A one-way valve may prevent blood from leaking or flowing from the right atrium through an implanted device, accordingly, an implant may remain in place for a long time, e.g., years.

In some embodiments, a percutaneous method is used for implanting a tube such that one of the tube's openings is inside the right atrium of the heart of a patient, and the other opening is on, or close to, the patient's skin. One or more catheters may be inserted, through an implanted tube, into the right atrium thus enabling procedures that require extracting blood from a patient, treating the blood (e.g., by an external system such as a dialysis machine or system) and returning the blood to the patient's vascular system.

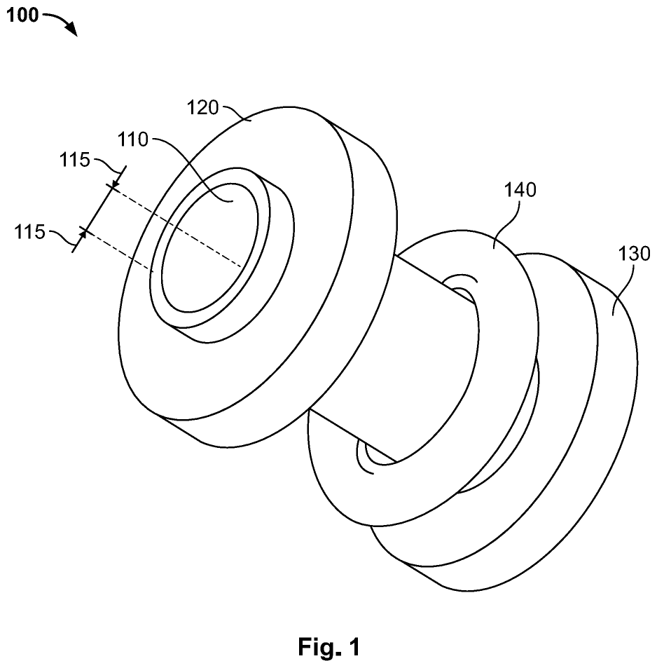

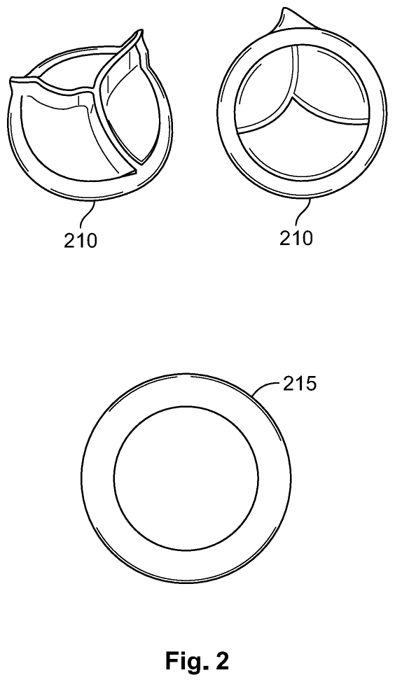

Reference is made to FIG. 1, which shows a device 100 according to illustrative embodiments of the present invention. As shown, device 100 may include a tube 110 having an internal diameter as illustrated by arrows 115, a first surface 120, a second surface 130 and a locking or placement unit 140. Reference additionally made to FIG. 2, which shows a top and bottom view of a one-way valve 210 and a sealing apparatus 215 according to illustrative embodiments of the present invention.

As shown, one-way valve may include several flexible overlapping diaphragms. For example, valve 210 and apparatus 215 may be placed inside tube 210 and apparatus 215 may be used to pushed valve 210 to a desired place along tube 110 and to further prevent leakage of blood from the right atrium. In some embodiments, apparatus 215 is designed to provide sealing around the edge of valve 210.

As shown, valve 210 may be an artificial heart valve that can be used as the one-way valve. For example, valve 210 may be placed at the end of tube 110, e.g., such that it is inside the right atrium. When a catheter is inserted into tube 110, the catheter pushes the leaves in valve 210 in order to reach the internal space or volume of the right atrium. When no catheter or other element is present inside tube 110, the leaves in valve 210 return to their normal position thus sealing passage through valve 210. For example, in some embodiments, one-way valve 210 prevents blood leaking when the dialyze tube is not connected to device 100.

Although an artificial heart valve is shown in FIG. 2 and discussed herein it will be understood that any one-way valve, e.g., check valve, clack valve, non-return valve, reflux valve or retention valve may be used without departing from the scope of the invention, therefore, the scope of the present invention is not limited by the type of one-way valve used.

For the sake of clarity and simplicity, the portions, parts or end of device 100 that are inside the right atrium are referred to as internal herein, and, the portions, parts or end of device 100 that are outside the right atrium or outside a patient's body are referred to as external herein.

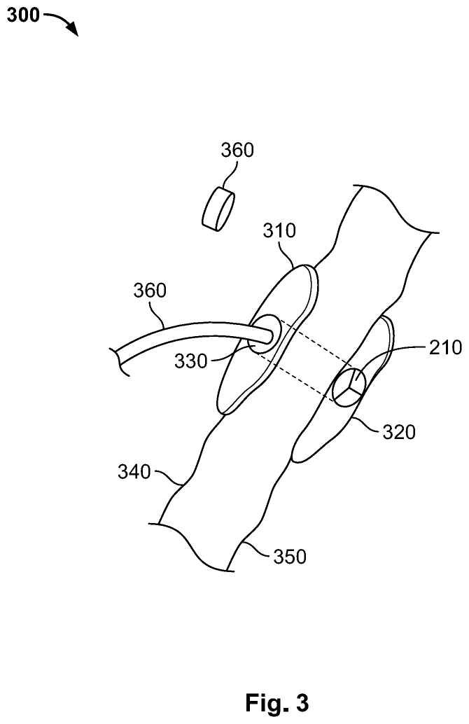

Reference is made to FIG. 3, which shows an implanted apparatus 300 and patient organs and tissues according to illustrative embodiments of the present invention. As shown, apparatus 300 may include an external part or surface 310 that may, in operation, be placed on a skin 340 of a patient. As shown, apparatus 300 may include an internal element, part or surface 320 that may, in operation, be placed or located inside the right atrium of the patient. For example, and as shown, internal surface may be placed on internal wall 350 that may be the internal wall of the right atrium. As further shown, one-way valve 210 may be located in, or near, the internal part of apparatus 300. As further shown, apparatus 300 may include a tube 330 that enables inserting a catheter 360 into the right atrium. Cap 360 may be used for hermetically sealing tube 330, for example, cap 360 may be screwed on an end or opening of tube 330. Catheter 360 may be a dialysis catheter with an outer diameter between 2.5 mm to 5.5 mm (11 to 16 French).

Some or even all parts of an apparatus 300 may be made of silicone or stainless steel. In some embodiments, a thin layer of Teflon is used for coating some parts of apparatus 300, for example, to reduce growing of soft tissue of germs. Any flexible polymer, e.g., FDA approved, can be used for coating or manufacturing the walls of tube 330. Rigid material may be used, for example, to prevent the wall of tube 330 from collapsing under pressure of surrounding tissues, stainless still may be used for manufacturing tube 330. Surfaces 310 and 320 may be designed and manufactured such that they do not include sharp edges, surfaces 310 and 320 may be made of flexible material and may be coated with Teflon® or similar material.

In some embodiments, one-way valve 210 may be designed such that it can accommodate dialysis catheters with an outer diameter between 11 to 16 French. In some embodiments, tube 330 may be narrow in the middle and wider towards its openings such that minimal stresses or pressure is applied to the heart wall.

Various methods may be used for placing or implanting device 300. For example, an endovascular catheter with stiff guiding sheath, in which a 16 to 19 Gauge needle can be housed, may be used in order to puncture the anterior wall of the right atrium and anterior chest wall soft tissues for the establishment of an initial tract. For example, a micro-puncture access assembly as known in the art may be used to puncture a hole in a patient's chest. Once through, a stiff, 0.035 inch wire is placed, over which dilatations with stiff dilators are performed to widen the hole created and enable placement of apparatus 300.

In some embodiments, apparatus 300 may be placed by inserting it, e.g., through the superior vena cava or the inferior vena cava, into the right atrium and pushing it through the wall of the right atrium until external surface 310 is in place (e.g., on the patient's skin). For example, an endovascular approach may be used whereby device 300 is brought to place from within the right atrium and not using percutaneous puncture of the chest wall towards the right atrium of the heart.

For example, a needle apparatus may be passed through an angled stiff guiding catheter within a stiff sheath, so that the anterior portion of the wall of the right atrium can be easily punctured on way out and towards the skin (e.g., in a way similar to an endoscopic puncture procedure that is performed by a gastroenterologist when placing feeding tubes in stomach). A procedure for placing device 300 may be performed according to an endovascular approach, e.g., device 300 may be brought to place via the right/left internal/external, upper extremity vein, jugular veins, subclavian veins, femoral veins, hepatic veins and Inferior vena cava, and pushed through the anterior wall of the right atrium, through the anterior chest wall. As described, the internal surface (e.g., one of disks 120 or 130 or internal surface 320) may limit movement of tube 330 so that once in place, external surface 310 (or the outer disc in device 100) can be slidden on the outer portion of tube 330 and locked in place, e.g., sown to skin 340.

In some embodiments, to lock device 300 in place, internal surface 320 is pressed against the internal wall of the right atrium (350) and external surface 310 is pressed against the patients skin 340. For example, surface 310 may be designed to move along tube 330 and be locked in place by notches, e.g., the way plastic handcuffs (or plasticuffs or flexicuffs) are locked. In another example, tube 330 may include grooves such that when surface 310 is rotated it is forced to move along tube 330 the way a nut moves along a bolt, thus locking apparatus 300 in place.

In some embodiments, e.g., as illustrated by FIG. 1, an elastic ring may be used for locking device 100 in place. For example, after device 100 is placed, ring 140 (that may be external to the patient) may be moved or slid along tube 110 and towards the skin of the patient, thus pulling the internal surface (e.g., surface 130 that may be inside the right atrium) against the right atrium's wall and locking device 100 in place. Although not shown, a second ring similar to ring 140 may be located inside the right atrium and may be pushed towards the internal wall of the right atrium.

Any other method or system may be used for placing, and/or locking or fixing, device 300 in place may be used, it will therefore be understood that the scope of the invention is not limited by the system or method used for placing or implanting device 300.

As described, a method of treating a patient may include placing, in the right atrium of the patient, a device including at least one one-way valve; and using the device for drawing blood from the right atrium, treating the blood and providing a liquid to the patient. For example, device 300 may be placed or implanted as described and catheters inserted via tube 330 may be used for drawing blood from a patient's right atrium, providing the drawn blood to a dialysis machine and returning blood from the dialysis machine to the patient's right atrium. In some embodiments, blood may be drawn from a patient using device 300 as described but returning blood or liquids to the patient may be done using a different system or method. For example, treated blood from a dialysis machine may be returned to a patient using any intravenous system or method as known in the art.

A device implanted in the right atrium as described may be designed to be hermetically sealed. For example, after removing catheters or tubes used in a dialysis procedure, cap 360 may be used to hermetically seal device 300 such that blood cannot leak through device 300 and such that undesirable substances cannot enter the patient's blood system via device 300. Accordingly, a device according to some embodiments enables a patient who requires periodic dialysis or other blood treatments to lead normal life, e.g., after a dialysis session is completed, device 300 may be sealed and the patient may be discharged.

In some embodiments, a device includes a conduit designed to house the one-way valve (e.g., tube 330) and at least one elastic ring mounted on the conduit and adapted to hold the conduit in place, e.g., ring 140 may hold or keep tube 110 in place as described herein.

A device may include a conduit designed to house the one-way valve, for example, tube 330 houses valve 210 as described, and the device may further include first and second surfaces mounted on the conduit and adapted to hold the conduit in place by applying pressure on the patient's skin and on the inner wall of the right atrium. For example, external surface 310 (first surface) and internal surface 320 (second surface) hold device 300 in place by respectively applying pressure on skin 340 and internal wall 350.

In some embodiments, a method for placing a percutaneous one-way valve device into the right atrium of the heart of a patient comprises the steps of: accessing the right atrium of the heart through an anterior chest wall of the patient, using a percutaneous needle; deploying a plurality of suture mounted anchors into the right atrium of the heart, through the percutaneous needle; anteriorly drawing the right atrium of the heart towards the anterior chest wall; inserting the percutaneous one-way valve device into the right atrium of the heart along a guidewire, wherein the one-way valve device comprises a one-way valve, an internal disc and an external disc.

In some embodiments, a method of deploying an apparatus includes accessing the right atrium of the heart through an anterior chest wall of a patient, using a percutaneous needle; deploying a plurality of suture mounted anchors into the right atrium of the heart, through the percutaneous needle; anteriorly drawing the right atrium of the heart towards the anterior chest wall; inserting the device into the right atrium of the heart along a guidewire; adjusting the distance between the first and second surfaces; tightening and/or locking the first and second surfaces in place; and removing the guidewire, the suture mounted anchors and the percutaneous needle. For example, to place device 100 suture mounted anchors are inserted, through a hole punctured in the patient's chest, into the right atrium of the heart and are used to pull internal wall 350 outwards, towards skin 340, next, device 300 may be inserted through the hole and surfaces 310 and 320 may be adjusted so that they respectively apply pressure to skin 340 and internal wall 350 thus locking device 300 in place. Otherwise described, skin 340 and internal wall 350 (and any tissues between skin 340 and internal wall 350) may be clamped by surfaces 310 and 320 thus holding and/or fixing device 300 in place.

Any device or assembly used for, or during, the placing or deployment procedure (e.g., suture mounted anchors, percutaneous needle etc.) may be removed once device 300 is securely placed. The percutaneous needle used as described may be a 19 Gauge needle. The guidewire used as described may be a 0.035 Inch guidewire.

In some embodiments, a method of performing dialysis via a one-way may include inserting a dialysis catheter into the one-way valve, e.g., a catheter (e.g., catheter 360) is inserted through tube 330 and through valve 210 located at the end of tube 330. Dialysis may be performed by a system connected to the catheter (e.g., a dialysis machine). When the dialysis procedure is finished, the catheter 360 may be removed and the one-way valve 210 may be flushed or otherwise cleaned and sealed, e.g., using cap 360 as described. For example, flushing of valve 210 and/or any other parts of device 300 may include using heparinized saline at a concentration of about 1000 units/cc.

An embodiment may provide guidance to a user (e.g., to a physician conducting a dialysis procedure) by using at least one of: real-time ultrasound imaging and a fluoroscopic guidance system. Any other imaging system or method may be used for providing a user with a required view, e.g., an internal view of the right atrium.

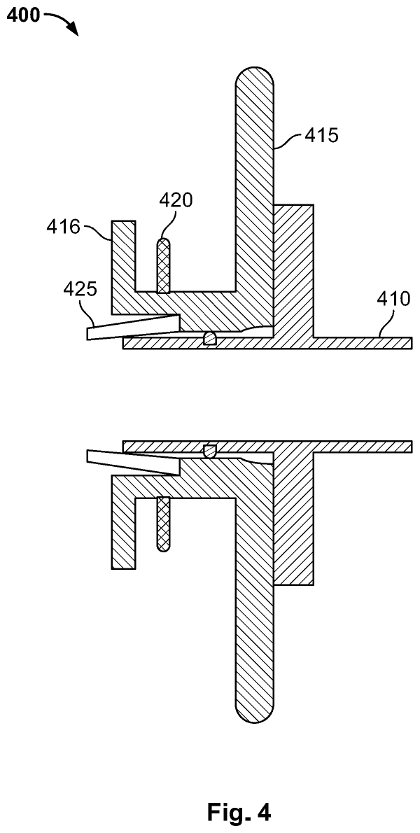

Reference is made to FIG. 4, which shows an implantable apparatus 400 according to illustrative embodiments of the present invention. As shown, apparatus 400 may include a main element or part that includes two surfaces 415 and 416. For example, surface 415 may be, or may be similar to, surface 310 and surface 416 may be similar to surface 320. As further shown, apparatus 400 may include a fastening ring 420 that may be similar to ring 140. Apparatus 400 may include one-way valve 425 that may be pushed open by guide 410. When apparatus 400 is placed or implanted, surface 416 may be located inside the right atrium and ring 420 may be pushed towards surface 416 such that the wall of the right atrium is clamped between ring 420 and surface 416 thus holding apparatus 400 in place. A second or additional ring (not shown) may be pushed towards surface 415 thus providing additional force that further secures apparatus 400 in place and/or sets the distance between the wall of the right atrium and the skin of the patient. In some embodiments, one-way valve is designed such that when guide 410 is pulled out of apparatus 400, one-way valve 425 closes (e.g., one-way valve 425 may be similar to one-way valve 210).

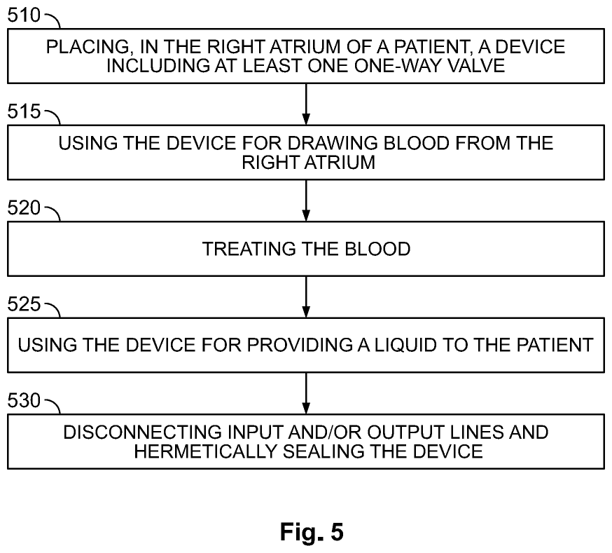

Reference is made to FIG. 5, which is a flowchart of a method according to illustrative embodiments of the present invention.

As shown by block 510, a device that includes a one-way valve may be placed, or implanted, in the right atrium of a patient. For example, device 100 may be placed or implanted as described such that a first opening of a tube or conduit in device 100 is inside the right atrium of a patient and a second opening of the tube or conduit is outside the patient's body, e.g., on the skin of the patient.

As shown by block 515, the device may be used for drawing blood from the patient. For example, when opened by a guide as described, one-way valve 210 may enable flow of liquids to/from the right atrium (e.g., to/from a dialyses machine). When closed, one-way valve 210 may prevent flow of blood from the right atrium.

As shown by block 520, blood drawn from the right atrium may be treated, e.g., by a dialysis machine as described. As shown by block 525, the device may be used for providing liquids to the patient. For example, liquids provided may include blood treated by a dialysis machine and/or any other solutions or fluids, e.g., replenishment solutions such as saline.

As shown by block 530, a method may include hermetically sealing the device after disconnecting input and/or output lines. For example, after a dialysis session, catheters or other lines may be removed, the device may be sealed, and the patient may be discharged. It is noted that in some embodiments, the device may be left implanted for weeks, months or even years.

It is noted that the term “about” may be used herein to include a range of ±10% of the disclosed value. It is further noted that the terms “dialysis” and “cardiac dialysis” are interchangeable throughout this document, unless specifically mentioned otherwise or unless it would have been obvious to a person skilled in the art that the terms are not equivalent in a specific instance. It is further noted that the terms “percutaneous one-way valve device” and “one-way valve device” are interchangeable throughout this document, unless specifically mentioned otherwise or unless it would have been obvious to a person skilled in the art that the terms are not equivalent in a specific instance.

In the description and claims of the present application, each of the verbs, “comprise” “include” and “have”, and conjugates thereof, are used to indicate that the object or objects of the verb are not necessarily a complete listing of components, elements or parts of the subject or subjects of the verb. Unless otherwise stated, adjectives such as “substantially”, “approximately” and “about” modifying a condition or relationship characteristic of a feature or features of an embodiment of the disclosure, are understood to mean that the condition or characteristic is defined to within tolerances that are acceptable for operation of an embodiment as described. In addition, the word “or” is considered to be the inclusive “or” rather than the exclusive or, and indicates at least one of, or any combination of items it conjoins.

Descriptions of embodiments of the invention in the present application are provided by way of example and are not intended to limit the scope of the invention. The described embodiments comprise different features, not all of which are required in all embodiments. Some embodiments utilize only some of the features or possible combinations of the features. Variations of embodiments of the invention that are described, and embodiments comprising different combinations of features noted in the described embodiments, will occur to a person having ordinary skill in the art. The scope of the invention is limited only by the claims.

While certain features of the invention have been illustrated and described herein, many modifications, substitutions, changes, and equivalents may occur to those skilled in the art. It is, therefore, to be understood that the appended claims are intended to cover all such modifications and changes as fall within the true spirit of the invention.

Various embodiments have been presented. Each of these embodiments may of course include features from other embodiments presented, and embodiments not specifically described may include various features described herein.

1. A method of treating a patient, the method comprising:
placing, in the right atrium of the patient, a device including at least one one-way valve; and
using the device for drawing blood from the right atrium, treating the blood and providing a liquid to the patient.

2. The method of claim 1, wherein the device is implanted in the right atrium and designed to be hermetically sealed when disconnected from input and output lines.

3. The method of claim 1, wherein the device includes:
a conduit designed to house the one-way valve; and
at least one elastic ring mounted on the conduit and adapted to hold the conduit in place.

4. The method of claim 1, wherein the device includes:
a conduit designed to house the one-way valve; and
first and second surfaces mounted on the conduit and adapted to hold the conduit in place by applying pressure on the patient's skin and on the inner wall of the right atrium.

5. The method of claim 4, comprising:
accessing the right atrium of the heart through an anterior chest wall of the patient, using a percutaneous needle;
deploying a plurality of suture mounted anchors into the right atrium of the heart, through the percutaneous needle;
anteriorly drawing the right atrium of the heart towards the anterior chest wall;
inserting the device into the right atrium of the heart along a guidewire;
adjusting the distance between the first and second surfaces;
tightening locking the first and second surfaces in place; and
removing the guidewire, the suture mounted anchors and the percutaneous needle.

6. The method of claim 5, wherein the percutaneous needle is a 19G needle.

7. The method of claim 5, wherein the guidewire is an 0.035 inch guidewire.

8. The method of claim 5, comprising performing dialysis via the one-way valve by:
inserting a dialysis catheter into the one-way valve;
performing dialysis by a system connected to the catheter;
flushing the one-way valve device;
removing the dialysis catheter; and
sealing the one-way valve.

7. The method of claim 8, wherein the flushing includes using heparinized saline at a concentration of about 1000 units/cc.

8. The method of claim 1, wherein the one-way valve is made of several flexible overlapping diaphragms.

9. The method of claim 1, wherein the at least one one-way valve is designed to be used with a dialysis catheter with an outer diameter between 2.5 mm to 5.5 mm.

10. The method of claim 1, comprising providing guidance to a user by using at least one of: real-time ultrasound imaging and a fluoroscopic guidance system.

11. The method of claim 1, wherein the device is placed via one of: an upper extremity vein, a lower extremity vein, a direct inferior vena cava (IVC) puncture and an hepatic vein.

12. The method of claim 1, wherein the device is placed in the right atrium using a micro-puncture access assembly.

13. An implantable apparatus for treating a patient, the apparatus comprising:
a tube housing a one-way valve and having first and second surfaces;
wherein when the apparatus is implanted, the first surface is located inside the right atrium of the patient and the second surface is located on the skin of the patient and wherein, when implanted, the apparatus is used for drawing blood from the right atrium, treating the blood and providing a liquid to the patient.

14. The apparatus of claim 13, wherein the apparatus is designed to be hermetically sealed when disconnected from input and output lines.

15. The apparatus of claim 13, wherein the first and second surfaces are adapted to hold the tube in place by respectively applying pressure to the patient's skin and to the inner wall of the right atrium.

16. The apparatus of claim 13, wherein the apparatus is used for performing dialysis by:
inserting a dialysis catheter through the one-way valve;
performing dialysis by a system connected to the catheter;
removing the dialysis catheter; and
sealing the one-way valve.

17. The apparatus of claim 13, wherein the apparatus is implanted by:
accessing the right atrium of the heart through an anterior chest wall of the patient, using a percutaneous needle;
deploying a plurality of suture mounted anchors into the right atrium of the heart, through the percutaneous needle;
anteriorly drawing the right atrium of the heart towards the anterior chest wall;
inserting a portion of the apparatus into the right atrium of the heart along a guidewire;
adjusting the distance between the first and second surfaces;
tight locking the first and second surfaces in place; and
removing the guidewire, the suture mounted anchors and the percutaneous needle.

18. The apparatus of claim 13, wherein the apparatus includes at least one elastic ring mounted on the tube and adapted to hold the conduit in place.

19. The apparatus of claim 13, wherein the apparatus is placed via one of: an upper extremity vein, a lower extremity vein, a direct inferior vena cava (IVC) puncture and an hepatic vein.

20. A method of treating a patient, the method comprising:
implanting a device that includes a conduit, the conduit housing a one-way valve, such that one opening of the conduit is inside the right atrium of the patient and another opening of the conduit enables inserting a tube into the right atrium; and
using the device for at least one of: drawing blood from the right atrium and providing a liquid to the patient.
